(19) United States
(12) Patent Application Publication
(10) Pub. No.: US 2015/0217076 A1
(43) Pub. Date: Aug. 6, 2015

(54) Endotracheal tube connector positioning system and method

(71) Applicants: Gary Steven Sichau, Chelmsford, MA (US); Terence Kernan Gray, Cape Elizabeth, ME (US)

(72) Inventors: Gary Steven Sichau, Chelmsford, MA (US); Terence Kernan Gray, Cape Elizabeth, ME (US)

(21) Appl. No.: 14/544,420

(22) Filed: Jan. 5, 2015

Related U.S. Application Data
(60) Provisional application No. 61/964,484, filed on Jan. 6, 2014.

(51) Int. Cl. A61M 16/08; A61M 16/04; A61M 16/00

(52) U.S. Cl. CPC A61M 16/085; A61M 16/0003; A61M 16/0051; A61M 16/04; A61M 2205/18; A61M 2230/432

(57) ABSTRACT

A detection system and method comprising a proximal end that connects to a ventilating device, a distal end that connects to a ventilating tube, a lumen between the proximal end and the distal end, a housing surrounding the lumen that couples the proximal end and the distal end, wherein at least a portion of the housing is transparent, and at least one detection element at least partially exposed to gases in the lumen, whereby continuously monitoring the proper positioning of the ventilating tube in an airway.

### RELATED APPLICATIONS

This application claims the benefit of U.S. provisional patent application No. 61/964,484 filed Jan. 06, 2014, the contents of which are incorporated by reference in their entirety.

### FIELD OF THE INVENTIVE CONCEPTS

The subject invention relates to medical devices. Specifically, the invention relates to devices for connecting endotracheal tubes with endotracheal ventilating systems and detecting the presence of substances within gases.

### BACKGROUND OF THE INVENTIVE CONCEPTS

Endotracheal tubes are used to provide reliable ventilation and oxygenation to patients with compromised breathing pathways. If a patient is not ventilating properly or suffers an airway blockage for more than four minutes where no oxygen reaches the patient's lungs, he or she will likely suffer brain damage. Furthermore, if the blockage continues for more than eight minutes, the patient will likely suffer brain death either through hypoxic brain damage or cardiopulmonary arrest. In cases where the patient's airway is blocked, the time it takes to establish and secure a reliable airway and restore oxygen flow to the patient's lungs is critical. Ventilating devices are well known in the prior art and have been disclosed in several patents, including: U.S. Pat. No. 3,640,282 issued to Kamen et al; U.S. Pat. No. 4,244,362 issued to Anderson; U.S. Pat. No. 4,685,457 issued to Donenfeld; U.S. Pat. No. 5,546,936 issued to Virag et al.; and U.S. Pat. No. 7,140,370 issued to Tresnak et al. Each incorporated by reference in its entirety.

There is currently a need for a mechanism and method for reliably confirming the presence of carbon-dioxide gas within an endotracheal tube to ensure proper ventilation of a patient without the need for manipulation of external sensing devices.

There is currently a need for a mechanism and method for reliably providing continual feedback confirming the correct positioning of the endotracheal tube within the patient's airway to ensure proper ventilation without the need for manipulation of external sensing devices.

There is currently a need for a mechanism and method for reliably confirming the presence of carbon-dioxide gas within an endotracheal tube while also confirming the correct positioning of the endotracheal tube within the patient's airway to ensure proper ventilation without the need for manipulation of external sensing devices.

There is currently a need for a mechanism and method that operates as a contiguous part of the ventilation circuit to minimize the manipulation of the endotracheal tube once the endotracheal tube has been correctly positioned in the patient's airway.

There is currently a need for a mechanism and method that continuously detects the presence of measureable concentrations of at least carbon-dioxide in gases expelled through the endotracheal tube from the patient's lungs without the need for manipulating external devices, whereby limiting the number of pieces in the ventilating circuit, limiting the manipulation of the endotracheal tube, and reducing the risk of dislodging the properly positioned endotracheal tube.

There is currently a need for a mechanism and method that operates as a contiguous part of the ventilation circuit to provide a visual and/or audio signal from a detection element to indicate the correct positioning of the distal end of the endotracheal tube within the patient's airway.

There is currently a need for a mechanism and method that operates to allow for the attachment and detachment of external devices through the use of a at least one port while continuously providing confirmation of the correct positioning of the endotracheal tube.

There is currently a need for a mechanism and method that allows for the introduction of gases and liquids into the endotracheal tube through the use of the at least one port.

### BRIEF SUMMARY OF THE INVENTION

During medical procedures where an endotracheal tube system is required, the endotracheal tube is inserted in a patient's airway to provide a conduit to secure a reliable breathing pattern through a secure pathway. In this manner, after either an attempted or successful direct laryngoscopy, the endotracheal tube is most commonly inserted into the patient's airway through the nose or mouth, past the epiglottis and larynx, and into the trachea. Proper insertion and positioning of the endotracheal tube within the patient's airway is critical and may take several attempts, as there is a chance that the endotracheal tube is inserted into the stomach instead of into the lungs. The presence of carbon-dioxide in the gases expelled from the lungs through the endotracheal tube is a primary method of determining if the endotracheal tube is correctly positioned within the patient's airway. Typically, exhaled carbon dioxide gas from a patient's lungs is between 30 mmHg and 40 mmHg (but can range at times between 20 mmHg and 80 mmHg or higher). If proper endotracheal tube placement and ventilation is achieved, the patient's exhaled gas has a measurable concentration of carbon dioxide. If the endotracheal tube is improperly placed within the esophagus there should be no sustained or significant concentration of carbon dioxide in the exhaled gas. A chemically treated material is typically used in lung ventilation procedures to assist in determining if the patient's exhaled gas contains significant levels of carbon dioxide, which indicates proper placement and ventilation. If the exhaled gas, either passing through or in close contact with the material, contains significant levels of carbon dioxide, the material turns from one color to another color. Typically the material turns from blue to yellow if significant levels of carbon dioxide are present in the exhaled gas. Carbon dioxide detectors used with ventilating devices are well known in the prior art and are disclosed by U.S. Pat. No. 6,427,687 issued to Kirk and U.S. Pat. No. 7,140,370 issued to Tresnak et al., each incorporated by reference in its entirety.

Once the endotracheal tube has been inserted into the patient's airway, a separate external device capable of sensing carbon-dioxide is attached, via a connector, to the proximal end of the endotracheal tube and the separate external sensing device then indicates whether or not carbon-dioxide is present in the gases expelled through the endotracheal tube. An absence of carbon-dioxide would indicate that upon insertion, the distal end of the endotracheal tube passed into the esophagus leading to the stomach instead of into the trachea. If this occurs, the endotracheal tube must be removed and reinserted until the distal end of the tube enters the trachea and the external sensing device indicates the presence of carbon-dioxide. Once the external sensing device detects carbon-dioxide and confirms the correct positioning of the endotracheal tube, the external sensing device is then detached from the connector, and a ventilation system is then attached to the proximal end of the endotracheal tube via the connector. Each time an external device is attached to or detached from the connector, there is a chance that the endotracheal tube is dislodged from its proper position in the trachea. In many situations successful reinsertion and positioning of the endotracheal tube in the trachea is either extremely difficult or impossible due to swelling of the trachea caused by the initial insertion of the endotracheal tube.

Once the endotracheal tube is correctly positioned within the patient's airway, it must remain in that position to ensure that the patient receives constant and sufficient airflow to the lungs. Any manipulation of the endotracheal tube after its insertion may cause the tube to be dislodged from an effective position, resulting in insufficient airflow and brain damage or death. Without constant reattachment and removal of the external sensing device, an action which in and of itself can cause the dislodgement of the endotracheal tube, it is difficult to determine if the tube is still positioned in an effective position. The presently used sensing devices are not only repeatedly attached and detached from the connector, but their cumbersome design does not afford them to be used as a permanent part of the ventilating circuit.

Therefore, a need exists to provide a mechanism and method for reliably assessing the presence of carbon-dioxide gas and providing continual feedback as to whether the endotracheal tube is correctly positioned within the patient's airway to ensure proper ventilation, without the need for reattaching and detaching a carbon-dioxide sensing device.

It is therefore an object of the present inventive concept to provide a new mechanism and method for connecting ventilation systems to the proximal end of an endotracheal tube.

It is a further object of the present inventive concept to provide such a mechanism and method which operates to continuously detect the presence of measureable concentrations of at least carbon-dioxide in the gas expelled through the endotracheal tube from the patient's lungs, without the need for attachment and detachment of peripheral devices, whereby limiting the number of pieces in the ventilating circuit, limiting the manipulation of the endotracheal tube and attached connector, and reducing the risk of dislodging the properly positioned endotracheal tube.

It is a further object of the present inventive concept to provide such a mechanism and method which operates as a contiguous part of the ventilation circuit to verify the correct positioning of the endotracheal tube within the trachea by detecting and measuring the concentration level of carbon dioxide in the gases exhaled through the endotracheal tube.

It is a further object of the present inventive concept to provide such a mechanism and method which operates as a contiguous part of the ventilation circuit to minimize the manipulation of the endotracheal tube once it has been correctly positioned in the patient's airway.

It is a further object of the present inventive concept to provide such a mechanism and method which operates as a contiguous part of the ventilation circuit to at least visually monitor a detection element for indicating at least a minimum predetermined concentration level of carbon dioxide in the gases exhaled through the endotracheal tube.

It is a further object of the present inventive concept to provide such a mechanism and method which operates as a contiguous part of the ventilation circuit to provide a visual and/or audio signal from a detection element to indicate the correct positioning of the distal end of the endotracheal tube within the patient's airway.

It is a further object of the present inventive concept to provide such a mechanism and method which operates to allow for the attachment and detachment of external devices through the use of a at least one port.

It is a further object of the present inventive concept to provide such a mechanism and method which operates to allow for the attachment and detachment of external devices for the monitoring and sensing of the gases within the lumen through the use of a at least one port.

It is a further object of the present inventive concept to provide such a mechanism and method which operates to allow for the introduction of gases and liquids into the endotracheal tube through the use of the at least one port.

In accordance with an aspect of the present inventive concepts, a detection system comprises a proximal end that connects to a ventilating device, a distal end that connects to a ventilating tube, a lumen between the proximal end and the distal end, a housing surrounding the lumen that couples the proximal end and the distal end and at least a portion of the housing is transparent, and at least one detection element at least partially exposed to gases in the lumen.

In an embodiment of the detection system, the at least one detection element is sensitive to at least one predetermined substance.

In an embodiment of the detection system, the at least one detection element is at least one chemically reactive material that changes from a first color to a second color to visually indicate the presence of the at least one predetermined substance in the lumen.

In an embodiment of the detection system, the at least one detection element comprises at least one electronic sensor that samples the gases in the lumen and emits at least one of at least one visual signaler or at least one auditory signaler to indicate the presence of the at least one predetermined substance in the lumen, a power source to power the electronic sensor, a power switch having an on position and an off position coupling the power source to the electronic sensor and moving the power switch to the on position activates the electronic sensor and moving the power switch to the off position deactivates the electronic sensor, and a power indicator coupled to the power source and the power switch to emit a predetermined visual signaler that indicates that the electronic sensor is activated.

In an embodiment of the detection system, the at least one visual signaler is at least one of a continuous emission of at least one colored light or an alternating emission of the at least one colored light.

In an embodiment of the detection system, the at least one auditory signaler is at least one predetermined auditory frequency and the at least one auditory signaler is at least one of a continuous emission of the at least one predetermined auditory frequency or an alternating emission of the at least one predetermined auditory frequency.

In an embodiment of the detection system, the at least one predetermined substance is carbon-dioxide.

In an embodiment of the detection system, the housing is at least partially transparent whereby providing visualization to at least one of the lumen or the at least one detection element.

In an embodiment of the detection system, the housing comprises at least one viewing port whereby providing visualization to at least one of the lumen or the at least one detection element.

In an embodiment of the detection system, the detection system further comprises at least one port wherein the at least one port comprises a first end coupled to the housing forming a first aperture whereby enabling transmission of at least one substance between the at least one port and the lumen, a second end having a second aperture configured and arranged to connect to at least one external device whereby enabling transmission of the at least one substance between the at least one port and the at least one external device, a port lumen between the first end and the second end, a port housing surrounding the port lumen that couples the first end and the second end, and a port cap removably coupled to the second end to close the second aperture.

In an embodiment of the detection system, the detection system further comprises at least one of a flexible distal extension, having at least one flexible distal joint, or a flexible proximal extension, having at least one flexible proximal joint, to decrease movement of the ventilating tube relative to movement of the housing.

In accordance with an aspect of the present inventive concepts, a detection method comprises providing a proximal end that connects to a ventilating device, providing a distal end that connects to a ventilating tube, providing a lumen between the proximal end and the distal end, providing a housing surrounding the lumen that couples the proximal end and the distal end, and providing at least one detection element at least partially exposed to gases in the lumen.

In an embodiment of the detection method, the at least one detection element is sensitive to at least one predetermined substance.

In an embodiment of the detection method, the at least one detection element is at least one chemically reactive material that changes from a first color to a second color to visually indicate the presence of the at least one predetermined substance in the lumen.

In an embodiment of the detection method, providing the at least one detection element comprises providing at least one electronic sensor that samples the gases in the lumen and emits at least one of at least one visual signaler or at least one auditory signaler to indicate the presence of the at least one predetermined substance in the lumen, providing a power source to power the electronic sensor, providing a power switch having an on position and an off position coupling the power source to the electronic sensor and moving the power switch to the on position activates the electronic sensor and moving the power switch to the off position deactivates the electronic sensor, and providing a power indicator coupled to the power source and the power switch to emit a predetermined visual signaler that indicates that the electronic sensor is activated.

In an embodiment of the detection method, the at least one visual signaler is at least one of a continuous emission of at least one colored light or an alternating emission of the at least one colored light.

In an embodiment of the detection method, the at least one auditory signaler is at least one predetermined auditory frequency and the at least one auditory signaler is at least one of a continuous emission of the at least one predetermined auditory frequency or an alternating emission of the at least one predetermined auditory frequency.

In an embodiment of the detection method, the at least one predetermined substance is carbon-dioxide.

In an embodiment of the detection method, the housing is at least partially transparent whereby providing visualization to at least one of the lumen or the at least one detection element.

In an embodiment of the detection method, the detection method further comprises providing at least one port wherein providing the at least one port comprises providing a first end coupled to the housing forming a first aperture whereby enabling transmission of at least one substance between the at least one port and the lumen, providing a second end having a second aperture configured and arranged to connect to at least one external device whereby enabling transmission of the at least one substance between the at least one port and the at least one external device, providing a port lumen between the first end and the second end, providing a port housing surrounding the port lumen that couples the first end and the second end, and providing a port cap removably coupled to the second end to close the second aperture.

In an embodiment of the detection method, the detection method further comprises providing at least one of a flexible distal extension, having at least one flexible distal joint, or a flexible proximal extension, having at least one flexible proximal joint, to decrease movement of the ventilating tube relative to movement of the housing.

In an embodiment, the detection system is separate and removable from the ventilating tube, whereby allowing for the proximal end of the ventilating tube to be cut and shortened.

In an embodiment, the detection system at least partially includes a carbon dioxide detection element used to sample the gases exhaled to assist in determining if the ventilating tube is positioned correctly to provide appropriate ventilation to the patient.

In an embodiment, the housing of the can be constructed of a transparent material or with a series of viewing ports to provide visualization of the lumen or the at least one detection element.

In an embodiment, the detection system includes at least one detection element at least partially contained within the housing.

In an embodiment, the detection system includes at least one detection element at least partially exposed to the gases within the lumen.

In an embodiment, the detection system includes at least one detection element at least partially exposed to the gases within the lumen where the detection element is sensitive to a predetermined gas within the lumen.

In an embodiment, the detection system includes at least one detection element at least partially exposed to the gases within the lumen, where the detection element measures the temperature of the gases within the lumen.

In an embodiment, the detection system further comprises at least one port through which the at least substance, such as gases or liquids, are introduced or extracted from the lumen without uncoupling the detection system from the ventilating device.

In an embodiment, the at least one port is configured and arranged to couple with the at least one external device to monitor temperature and chemical composition of the gases within the lumen without uncoupling the detection system from the ventilating device.

In an embodiment, at least one of the distal end or the proximal end of the detection system is elongated and includes a flexible elbow, whereby allowing physical manipulation of the detection system without moving the ventilation tube from its proper position.

### BRIEF DESCRIPTION OF THE DRAWINGS

The foregoing and other objects, features and advantages of the inventive concepts will be apparent from the more particular description of preferred embodiments of the inventive concepts, as illustrated in the accompanying drawings in which like reference characters refer to the same parts throughout the different views. The drawings are not necessarily to scale, emphasis instead being placed upon illustrating the principles of the inventive concepts. Other objects, features, and advantages will occur to those skilled in the art from the following description of a preferred embodiment and the accompanying drawings. In the drawings,

FIG. 1 shows a perspective view of a conventional connector coupled to a conventional ventilating tube inserted into an airway, shown in a cutaway perspective view.

FIG. 2 shows a perspective view of a conventional connector coupled to a conventional ventilating tube.

FIG. 3 shows a perspective view of an embodiment of a detection system.

FIG. 4 shows a perspective view of an embodiment of the detection system.

FIG. 5 shows a perspective view of an embodiment of the detection system including a viewing port.

FIG. 6 shows a perspective view of an embodiment of the detection system including an electronic sensor.

FIG. 7 shows a perspective view of an embodiment of the detection system including an electronic sensor and power mechanisms.

FIG. 8 shows a perspective view of an embodiment of the detection system including at least one port.

FIG. 9 shows a perspective view of an embodiment of the detection system including a flexible distal extension.

FIG. 10 shows a perspective view of an embodiment of the detection system including a flexible proximal extension.

### DETAILED DESCRIPTION OF THE INVENTION

The accompanying drawings are described below, in that example embodiments in accordance with the present inventive concepts are shown. Specific structural and functional details disclosed herein are merely representative. This invention may be embodied in many alternate forms and should not be construed as limited to example embodiments set forth herein.

Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present inventive concepts. As used herein, the term “and/or” includes any and all combinations of one or more of the associated listed items.

It will be understood that when an element is referred to as being “on,” “connected to” “abutting,” “coupled to,” or “extending from” another element, it can be directly on, connected to, abutting, or coupled to the other element or intervening elements may be present. In contrast, when an element is referred to as being “directly on,” “directly connected to,” “directly abutting,” “directly coupled to,” or “directly extending from” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

Aside from the preferred embodiment or embodiments disclosed below, this invention is capable of other embodiments and of being practiced or being carried out in various ways. Thus, it is to be understood that the invention is not limited in its application to the details of construction and the arrangement of components set forth in the following description or illustrated in the drawings. If only one embodiment is described herein, the claims hereof are not to be limited to that embodiment. Moreover, the claims are not to be read restrictively unless there is clear and convincing evidence manifesting a certain exclusion, restriction or disclaimer.

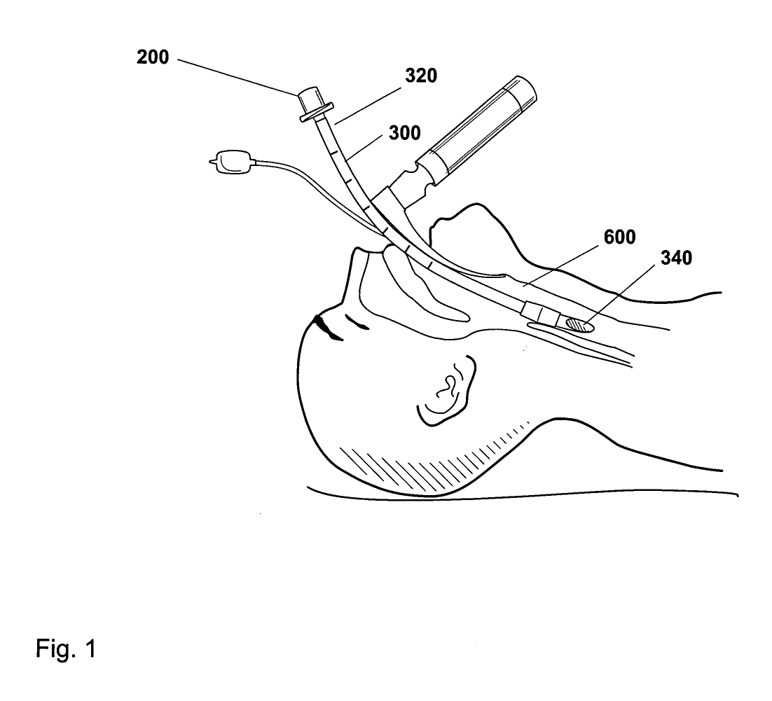

FIG. 1 shows a perspective view of a ventilating tube 300 inserted into an airway 600, shown in a cutaway perspective view. The ventilating tube 300, having a proximal tube end 320 and a distal tube end 340, is coupled to a conventional connector 200. Proper positioning of the ventilating tube 300 enables carbon-dioxide from the lungs to flow from the distal tube end 340 of the ventilating tube 300 to the proximal tube end 320.

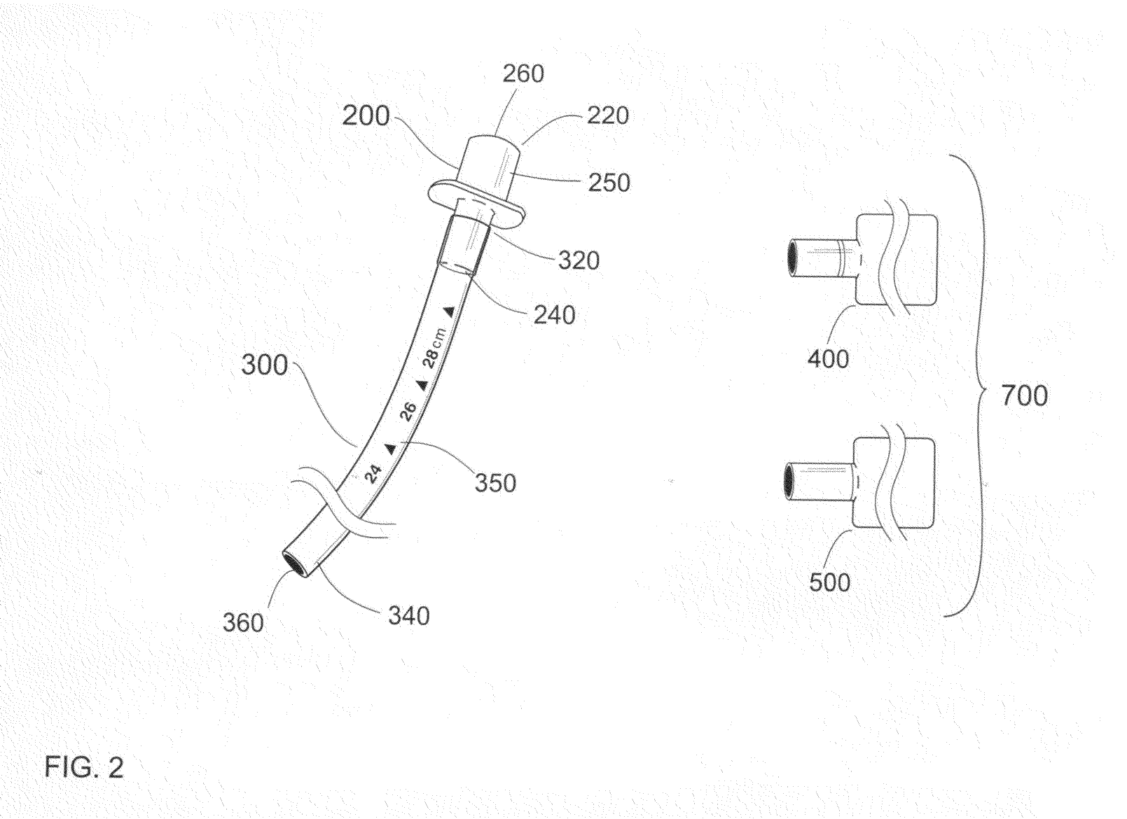

FIG. 2 shows a perspective view of a ventilating tube 300 and a conventional connector 200. The conventional connector 200 is separate from the ventilating tube 300 and connects the ventilating tube 300 to a ventilating device 400. The proximal tube end 320 and the distal tube end 340 of the ventilating tube 300 are connected by a tube housing 150 that surrounds a tube lumen 160. The conventional connector 200 has a proximal connector end 220 and a distal connector end 240 connected by a connector housing 250 that surrounds a connector lumen 260. The distal connector end 240 of the conventional connector 200 attaches to the tube proximal end 120 of the ventilating tube 300, and the proximal connector end 220 of the conventional connector 200 attaches to the ventilating device 400 or at least one external device 600, for example, a carbon-dioxide detector 500.

Once the ventilating tube 300 is properly positioned in the airway 600, the distal end 240 of the conventional connector 200 is inserted into the proximal tube end 320 of the ventilating tube 300. The carbon-dioxide detector 500 is then connected to the proximal connector end 220 of the conventional connector 200. Gases 800 expelled through the ventilating tube 300 and conventional connector 200 are analyzed by the carbon-dioxide detector 500 for the presence of carbon-dioxide gas to confirm proper positioning of the distal tube end 340 of the ventilating tube 300. If carbon-dioxide gas is detected, this indicates that the ventilating tube 300 is properly positioned and the carbon-dioxide detector 500 is then detached from the conventional connector 200 and then the ventilating device 400 is attached to the proximal connector end 220 of the conventional connector 200.

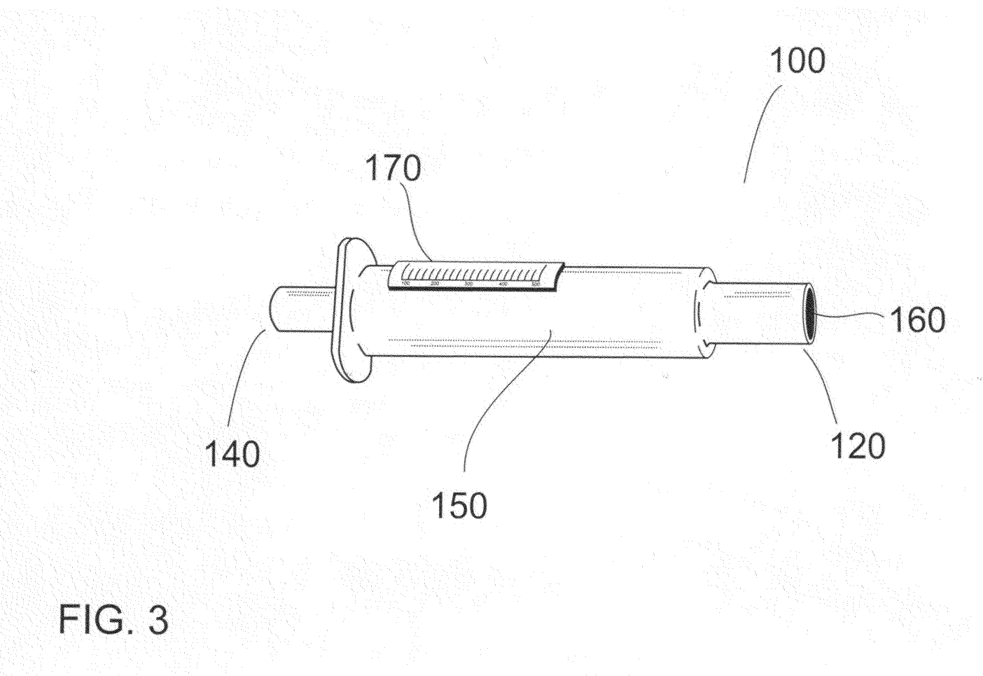

FIG. 3 shows a perspective view of an embodiment of a detection system 100 in accordance with aspects of the present invention. The detection system 100 comprises a proximal end 120, a distal end 140, a housing 150, a lumen 160 and at least one detection element 170. The housing 150 couples the proximal end 120 and the distal end 140, and surrounds the lumen 160. The housing 150 is at least partially transparent whereby providing visualization to the lumen 160, and the detection element 170 is arranged and configured within the housing 150 to at least partially expose the detection element 170 to gases 800 at the lumen 160.

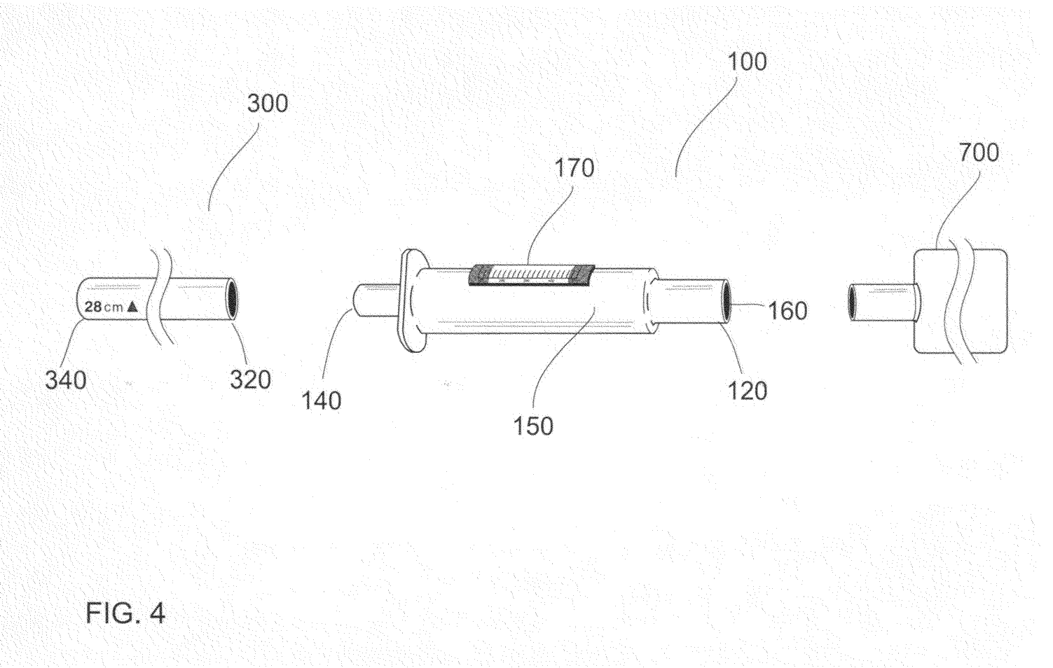

FIG. 4 shows a perspective view of an embodiment of the detection system 100 in accordance with aspects of the present invention. The detection system 100 comprises the proximal end 120, the distal end 140, the housing 150, the lumen 160 and the at least one detection element 170. The distal end 140 of the detection system 100 attaches to the proximal tube end 320 of the ventilating tube 300, and the proximal end 120 of the detection system 100 attaches to at least one external device 700. The external device may be the ventilating device 400 or the carbon-dioxide detector 500 as shown in FIG. 2, or may be any other device that is coupled to the ventilating tube 300. The detection system 100 is separable from the ventilating tube 300 to enable the ventilating tube 300 to be cut or shortened. Shortening the ventilating tube 300 may be necessary to aid manipulation of the ventilating tube 300. After the ventilating tube 300 is cut, the detection system can be recoupled to the shortened proximal tube end 320.

In an embodiment, the detection system 100 is combined with the ventilating tube 300 to form an inseparable single unit.

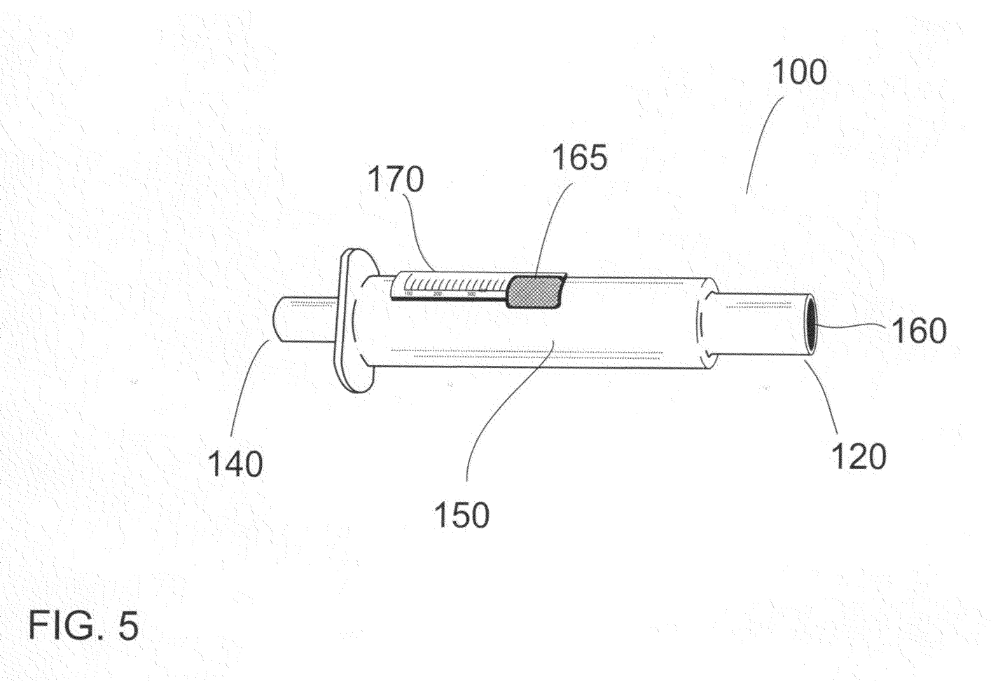

FIG. 5 shows a perspective view of an embodiment of the detection system 100, wherein the detection element 170 comprises at least one chemically reactive material 172 that is sensitive to at least one predetermined substance 810, for example carbon-dioxide, and wherein the chemically reactive material 172 is observable through the housing 150. The chemically reactive material 172 changes from a first color 174 to a second color 176 when the chemically reactive material 172 is exposed to the predetermined substance 810 at the lumen 160, whereby indicating the presence of the predetermined substance 810 at the lumen 160. The housing 150 is at least partially transparent whereby providing visualization to changes in color of the chemically reactive material 172.

In an embodiment, at least one chemically reactive material 172 is at least partially formed into the housing 150 and is at least partially exposed to the gases 800 present at the lumen 160.

As further shown in FIG. 5, the detection system 100 further comprises at least one viewing port 165 constructed into the housing 150 whereby providing unobstructed visualization to at least one of the lumen 160 or the detection element 170.

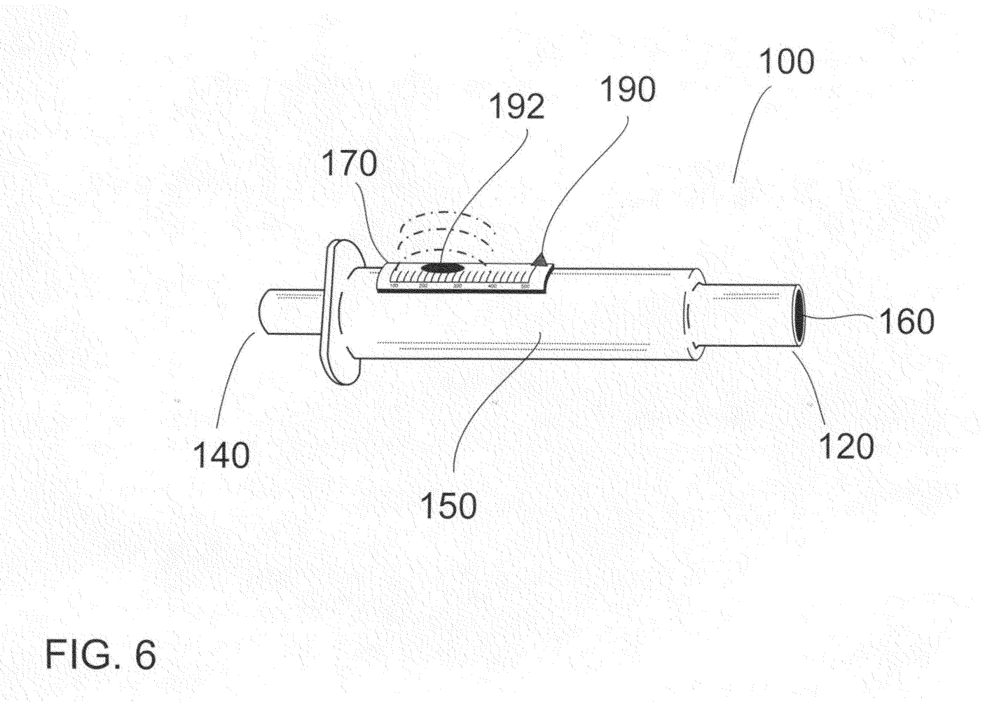

FIG. 6 shows a perspective view of an embodiment of the detection system 100, wherein the detection element 170 comprises at least one electronic sensor 180 at least partially exposed to the gases 800 at the lumen 160. The electronic sensor 180 samples the gases 800 and indicates whether the presence of the predetermined substance 810 is detected at the lumen 160. If the electronic sensor 180 detects the presence of the predetermined substance 810 at the lumen 160, the electronic sensor 180 emits at least one of at least one auditory signal 192 or at least one visual signal 190 to indicate the presence of the at least one predetermined substance 810 within the lumen 160. The electronic sensor 180 is programmed to detect the predetermined substance 810 and is programmed to emit the auditory signal 192 and/or the visual signal 190 continuously or in an alternating pattern.

In an embodiment, the electronic sensor 180 utilizes a light emitting diode to emit the visual signal.

Referring to FIG. 1, the distal tube end 340 of the ventilating tube 300 is positioned within the airway 600. The detection element 170 of the detection system 100, as shown in FIG. 6, samples the gases 800 within the lumen 160 and indicates the presence of the predetermined substance 810 at the lumen by emitting the auditory signal 192 and/or the visual signal 190. Emission of the auditory signal 192 and/or the visual signal 190 indicates that the distal tube end 340 of the ventilating tube 300 is properly positioned. After the detection element 170 indicates proper positioning of the distal tube end 340 of the ventilating tube 300, the ventilating device 400 is then attached to the proximal end 120 of the detection system 100, as shown in FIG. 4. The detection system 100 remains coupled between the ventilating device 400 and the ventilating tube 300, whereby providing a continuous visual and/or audio confirmation that the ventilating tube 300 is properly positioned without the need to continuously attach and detach the carbon-dioxide detector 500. This reduces the risk of dislodging the distal tube end 340 of the ventilating tube 300 from its proper position in the airway 600.

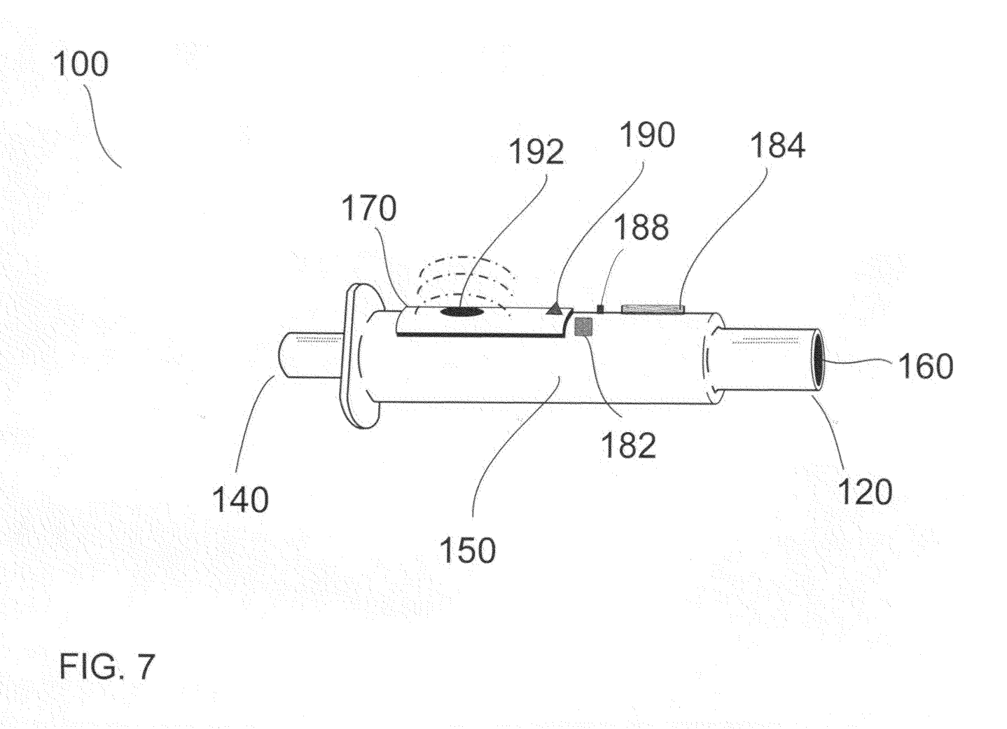

FIG. 7 shows a perspective view of an embodiment of the detection system 100, wherein the electronic sensor 180 further comprises a power source 182, a power switch 184, and a power indicator 188. The power switch is coupled between the power source 182 and the electronic sensor 180. The power source 182 powers the electronic sensor 180 when the power switch 184 is in an on position 185, and does not power the electronic sensor 180 when the power switch is in an off position 186. Powering the electronic sensor 180 enables the electronic sensor to sample the gases 800 at the lumen 160, and enables the electronic sensor 180 to emit the auditory signal 192 and/or the visual signal 190. The power indicator 188 is coupled to the power source 182 and the power switch 184, and the power indicator 188 is activated when the power switch 184 is moved from the off position 186 to the on position 185, whereby indicating that the electronic sensor 180 is activated.

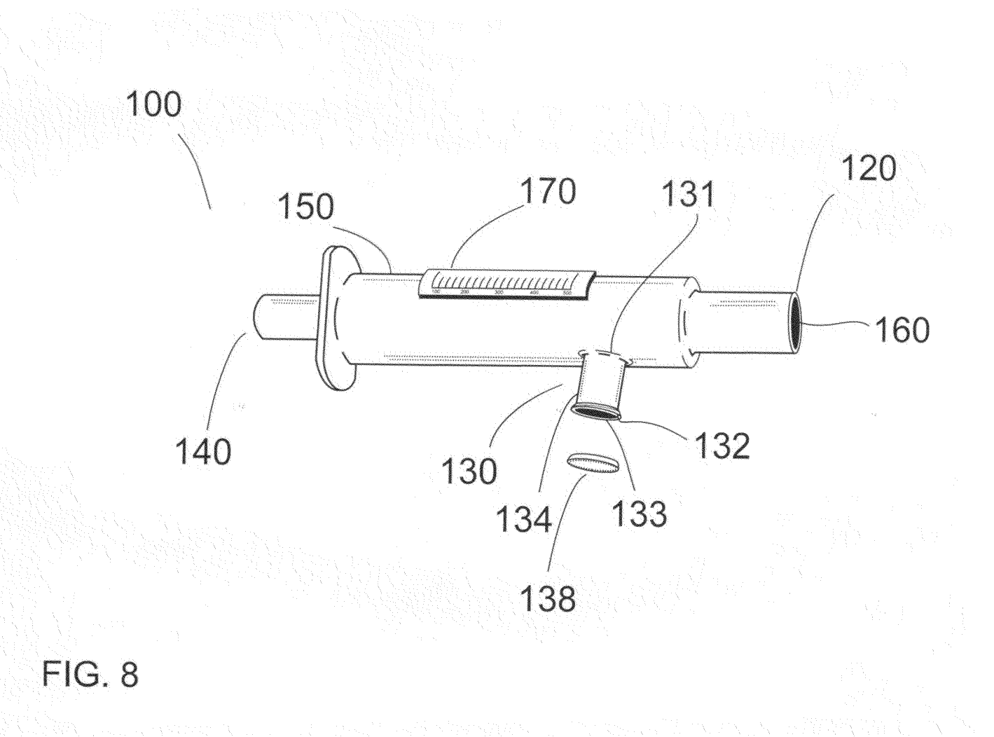

FIG. 8 shows a perspective view of an embodiment of the detection system 100, wherein the housing 150 comprises at least one port 130. The port 130 comprises a first end 131, a second end 132, a port housing 134, a port lumen 133, and a port cap 138. The first end 131 and the second end 132 are coupled together by the port housing 134 that surrounds the port lumen 133, and the port cap 138 is removably coupled to the second end 132 to seal a second aperture 136 of the second end 132. The first end 131 is coupled to the housing 150 forming a first aperture 135 whereby enabling transmission of at least one substance 820 between the port 130 and the lumen 160. The port cap 138 is removably coupled to the second aperture 136 to enable the at least one external device 700, shown in FIG. 2, to couple with the detection system 100. Removing the port cap 138 and coupling the external device 700 to the port 130 enables the at least one substance 820 to be introduced from the external device 700 to the detection system 100 or vice versa. Sealing the port cap 138 enables the detection system 100 to function as a closed system when the external device 700 is not needed. The port 130 is configured and arranged to couple with the external device 700 when the port cap 138 is uncoupled from the second end 132.

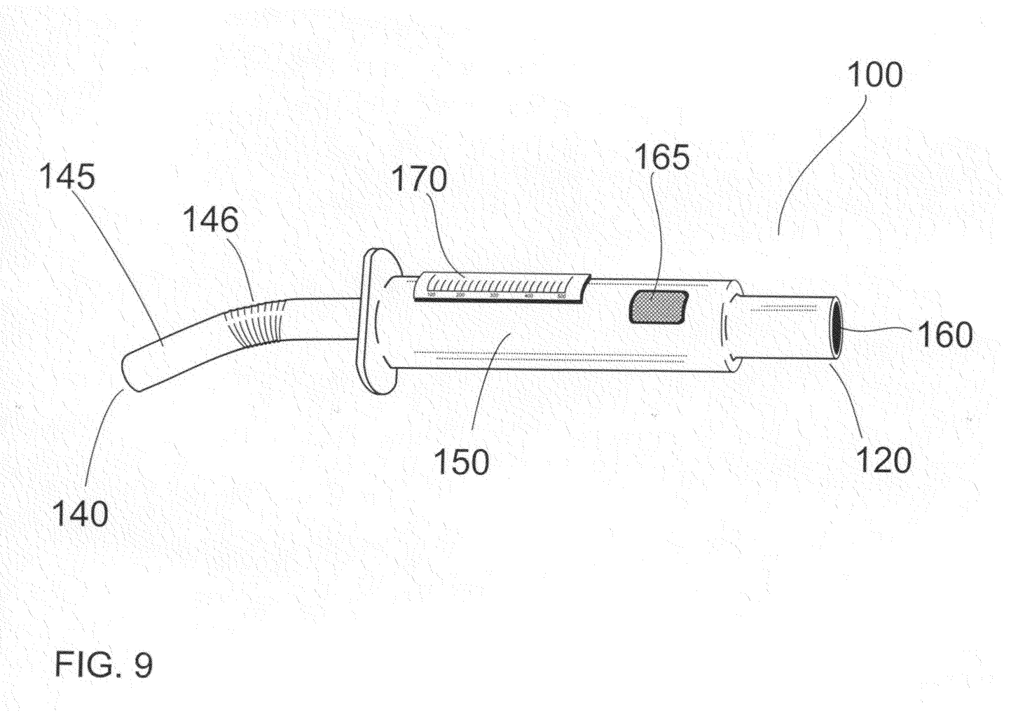

FIG. 9 shows a perspective view of an embodiment of the detection system 100 comprising a flexible distal extension 145 having the distal end 140 and at least one flexible distal joint 146. The flexible distal extension 145 and the flexible distal joint 146 enable the detection system 100 to be manipulated relative to the ventilating tube 300 with reduced risk of dislodging the distal tube end 340 of the ventilating tube 300 from the proper position within the airway, as shown in FIG. 1.

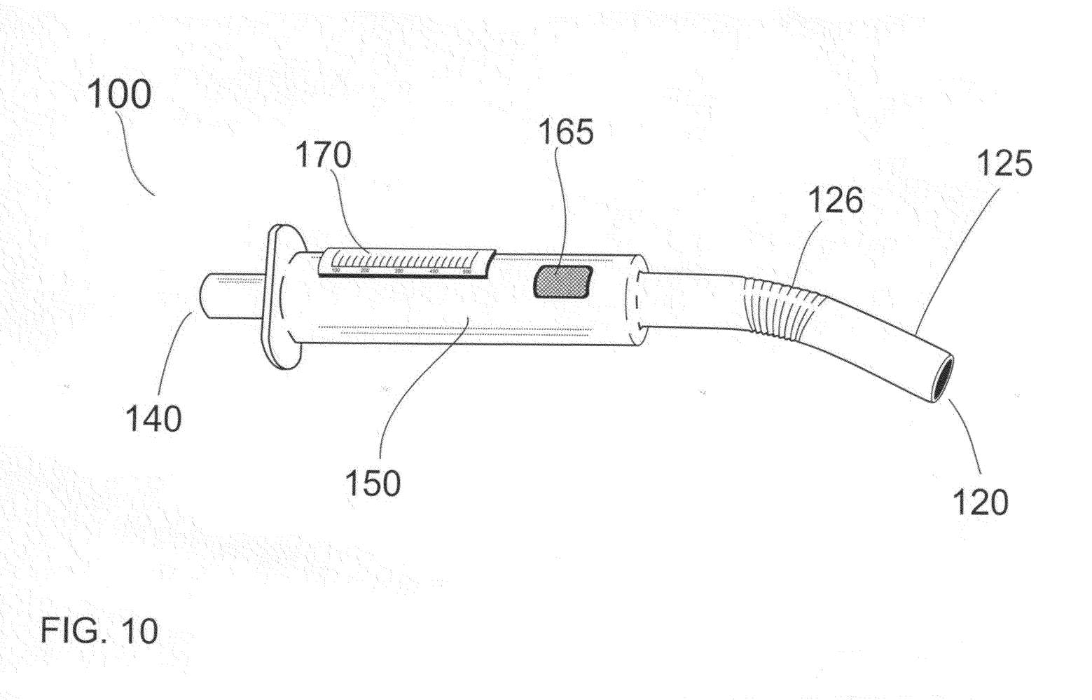

FIG. 10 shows a perspective view of an embodiment of the detection system 100 comprising a flexible proximal extension 125 having the proximal end 120 and at least one flexible proximal joint 126. The flexible proximal extension 125 and the flexible proximal joint 146 enable the detection system 100 to be manipulated relative to the ventilating tube 300, whereby reducing the risk of dislodging the distal tube end 340 of the ventilating tube 300 from the proper position within the airway, as shown in FIG. 1.

In an embodiment, the detection system 100 comprises the flexible distal extension 145, shown in FIG. 9, and the flexible proximal extension 125, shown in FIG. 10, whereby further reducing the risk of dislodging the distal tube end 340 of the ventilating tube 300 from the proper position within the airway, as shown in FIG. 1.

The foregoing inventive concepts may be embodied in many alternate forms and should not be construed as limited to example embodiments set forth herein. Accordingly, specific embodiments are shown by way of example in the drawings. It should be understood that there is no intent to limit the inventive concepts to the particular forms disclosed, but on the contrary, the inventive concepts are to cover all modifications, equivalents, and alternatives falling within the spirit and scope of the claims. Although specific features of the inventive concepts are shown in some drawings and not in others, this is for convenience only as each feature may be combined with any or all of the other features in accordance with the invention. The words “including”, “comprising” “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed, considering those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended. Other embodiments will occur to those skilled in the art and are within the following claims.

What is claimed is:

1. A detection system comprising:
a proximal end that connects to a ventilating device;
a distal end that connects to a ventilating tube;
a lumen between the proximal end and the distal end;
a housing, surrounding the lumen, that couples the proximal end and the distal end, wherein at least a portion of the housing is transparent; and
at least one detection element at least partially exposed to gases in the lumen.

2. The detection system of claim 1 wherein the at least one detection element is sensitive to at least one predetermined substance.

3. The detection system of claim 2 wherein the at least one detection element comprises at least one chemically reactive material that changes from a first color to a second color to visually indicate the presence of the at least one predetermined substance in the lumen.

4. The detection system of claim 2 wherein the at least one detection element comprises:
at least one electronic sensor that samples the gases in the lumen and emits at least one of at least one visual signaler or at least one auditory signaler to indicate the presence of the at least one predetermined substance in the lumen;
a power source to power the electronic sensor;
a power switch, having an on position and an off position, coupling the power source to the electronic sensor, and moving the power switch to the on position activates the electronic sensor and moving the power switch to the off position deactivates the electronic sensor; and
a power indicator, coupled to the power source and the power switch, to emit a predetermined visual signaler that indicates that the electronic sensor is activated.

5. The detection system of claim 4 wherein the at least one visual signaler is at least one of a continuous emission of at least one colored light or an alternating emission of the at least one colored light.

6. The detection system of claim 4 wherein the at least one auditory signaler is at least one predetermined auditory frequency, and the at least one auditory signaler is at least one of a continuous emission of the at least one predetermined auditory frequency or an alternating emission of the at least one predetermined auditory frequency.

7. The detection system of claim 2 wherein the at least one predetermined substance is carbon-dioxide.

8. The detection system of claim 2 wherein the housing comprises at least one viewing port whereby providing visualization to at least one of the lumen or the at least one detection element.

9. The detection system of claim 1 further comprising at least one port, wherein the at least one port comprises:
a first end coupled to the housing forming a first aperture, whereby enabling transmission of at least one substance between the at least one port and the lumen;
a second end, having a second aperture, configured and arranged to connect to at least one external device, whereby enabling transmission of the at least one substance between the at least one port and the at least one external device;
a port lumen between the first end and the second end;
a port housing, surrounding the port lumen, that couples the first end and the second end; and
a port cap, removably coupled to the second end, to close the second aperture.

10. The detection system of claim 1 further comprising at least one of a flexible distal extension, having at least one flexible distal joint, or a flexible proximal extension, having at least one flexible proximal joint, to decrease movement of the ventilating tube relative to movement of the housing.

11. A detection method comprising:
providing a proximal end that connects to a ventilating device;
providing a distal end that connects to a ventilating tube;
providing a lumen between the proximal end and the distal end;
providing a housing, surrounding the lumen, that couples the proximal end and the distal end, wherein at least a portion of the housing is transparent; and
providing at least one detection element at least partially exposed to gases in the lumen.

12. The detection method of claim 11 wherein the at least one detection element is sensitive to at least one predetermined substance.

13. The detection method of claim 12 wherein the at least one detection element is at least one chemically reactive material that changes from a first color to a second color to visually indicate the presence of the at least one predetermined substance in the lumen.

14. The detection method of claim 12 wherein providing the at least one detection element comprises:
providing at least one electronic sensor that samples the gases in the lumen and emits at least one of at least one visual signaler or at least one auditory signaler to indicate the presence of the at least one predetermined substance in the lumen;
providing a power source to power the electronic sensor;
providing a power switch, having an on position and an off position, coupling the power source to the electronic sensor, and moving the power switch to the on position activates the electronic sensor and moving the power switch to the off position deactivates the electronic sensor; and
providing a power indicator, coupled to the power source and the power switch, to emit a predetermined visual signaler that indicates that the electronic sensor is activated.

15. The detection method of claim 14 wherein the at least one visual signaler is at least one of a continuous emission of at least one colored light or an alternating emission of the at least one colored light.

16. The detection method of claim 14 wherein the at least one auditory signaler is at least one predetermined auditory frequency, and the at least one auditory signaler is at least one of a continuous emission of the at least one predetermined auditory frequency or an alternating emission of the at least one predetermined auditory frequency.

17. The detection method of claim 12 wherein the at least one predetermined substance is carbon-dioxide.

18. The detection method of claim 12 wherein the housing comprises at least one viewing port whereby providing visualization to at least one of the lumen or the at least one detection element.

19. The detection method of claim I further comprising providing at least one port, wherein providing the at least one port comprises:
providing a first end coupled to the housing forming a first aperture, whereby enabling transmission of at least one substance between the at least one port and the lumen;
providing a second end, having a second aperture, configured and arranged to connect to at least one external device, whereby enabling transmission of the at least one substance between the at least one port and the at least one external device;
providing a port lumen between the first end and the second end;
providing a port housing, surrounding the port lumen, that couples the first end and the second end; and
providing a port cap, removably coupled to the second end, to close the second aperture.

20. The detection method of claim 11 further comprising providing at least one of a flexible distal extension, having at least one flexible distal joint, or a flexible proximal extension, having at least one flexible proximal joint, to decrease movement of the ventilating tube relative to movement of the housing.
